SEQUENCE LISTING (1 sequence)

SEQ ID NO: 1 - protein, 535 aa, synthetic construct
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

(19) United States
(12) Patent Application Publication
(10) Pub. No.: US 2026/0002187 A1
(43) Pub. Date: Jan. 1, 2026

(54) AUTOMATED PERIPLASMIC EXTRACTION

(71) Applicant: Satorius Xpress Biologics, Herstal (BE)

(72) Inventors: Marc Daukandt, Bilstain (BE); Philippe Ledent, Liège (BE); Christian Rodriguez, Spa (BE)

(21) Appl. No.: 18/846,186

(22) PCT Filed: Mar. 20, 2023
(86) PCT No.: PCT/EP2023/057033

§ 371 Date: Sep. 11, 2024

(30) Foreign Application Priority Data
Mar. 18, 2022 (BE) 2022/5194

(51) Int. Cl. C12P 21/02; C07K 14/34

(52) U.S. Cl. CPC C12P 21/02; C07K 14/34

(57) ABSTRACT

Automated method for extracting a peptide of interest located in the periplasmic space and/or comprising an acidification step.

### TECHNICAL FIELD

The present invention relates to the automated extraction of a peptide of interest whose production has been induced in the periplasmic space.

### PRIOR ART

The production of a protein of interest in the periplasmic space is known. This has the advantage of establishing a first barrier with cytosolic contaminants including genomic DNA.

In practice, the external membrane of a Gram-negative bacterium, for example Escherichia coli, or Pseudomonas sp., is selectively broken, but not the internal membrane, which allows the contents of the periplasmic space to be released into the medium. Then, simple centrifugation allows the cells to be removed with all the contaminants found therein. Osmotic shock, where the cells are conditioned in a hypertonic medium (e.g. sucrose 20-25% by weight, at a neutral or alkaline pH), then suddenly placed in contact with a large volume of water, is commonly used for this purpose, but always at neutral or alkaline pH.

This technique has, however, several disadvantages and limitations. The first difficulty is that the internal membrane must not be broken, which limits the solutions that can be used, the treatment times, as well as the mechanical stresses that can be applied to these weakened cells. The second difficulty, inversely, is that the external membrane must be sufficiently broken, which introduces, on the contrary, fairly strict conditions.

In addition, peptides that would be absorbed into the membranes, or even inserted into them, risk not being well recovered, or could even weaken the internal membrane, which has been reported as problematic in the case of acidification when diphtheria toxin is produced in the periplasmic space of Escherichia coli (O'Keefe and Collier, PNAS Vol. 86, pp 343-346, 1989).

Another limitation is the difficulty of scaling-up: indeed, some peptides have to be mass-produced, which requires being able to process large quantities of cells while ensuring substantially identical processing conditions for all cells: lack of homogeneity or variable processing times are problematic. Thus, the use of larger containers promotes local inhomogeneities, which directly affects (i) the recovery yields of the peptide of interest, due, for example, to the poor breakage of the external membrane (osmotic shock too weak), and (ii) the purity, due to the undesired breakage of the internal membrane (e.g. osmotic shock too strong). In addition, the mixing and mechanical stirring means are limited, or there is a risk of breaking the internal membranes or even shearing the genomic DNA, which would render the batch completely unusable.

Some suggested improvements include the addition of salts or chaotropic molecules such as, among others, alkali-or alkaline-earth chlorides or sulphates, sodium phosphate, ammonium acetate, ammonium chloride, various detergents (sodium deoxycholate, Octyl-glucoside, Tween@80, Triton X-100), guanidinium chloride or urea, so as to further weaken the external membrane. However, as mentioned above, these molecules also risk further weakening the internal membrane, especially in the context of solutions that are difficult to homogenise.

WO 2008/088757 proposed a continuous extraction of the periplasmic content, where, as in batch mode, the cells are equilibrated in a hypertonic solution. This suspension is then pumped and, thanks to a T-system, water is added to it in large quantities via a second pump, so as to cause an osmotic shock, before centrifuging the mixture. The mixing of the two flows is done exclusively by static means, and the pH of the solutions is 7.2. A yield of approximately 70% is mentioned. However, the time during which the cells are in contact with the water is not described, nor is the purity of the protein of interest.

### BRIEF SUMMARY OF THE INVENTION

This invention relates firstly to a method for the automated extraction of a peptide of interest artificially secreted into the periplasmic space of a Gram-negative bacterium comprising the successive steps of:
suspending said bacteria in a hypertonic solution,
pumping said suspension in a hypertonic medium into a first mixing chamber, at a constant and calibrated flow rate Q1;
simultaneously pumping at a constant and calibrated flow rate Q2 a hypotonic solution into said mixing chamber, so that the mixture containing said cells, the hypertonic solution and the hypotonic solution remains for at least 10 seconds in said mixing chamber, so as to ensure breakage of the external membrane of said bacteria by osmotic shock while preserving the integrity of the internal membrane of said bacteria;
pumping said mixture to draw it from said mixing chamber towards a pipe, at a constant and calibrated flow rate Q3, so that the volume in said first mixing chamber makes it possible to ensure the desired residence time in said first mixing chamber, said pipe having a length and a diameter so as to ensure a contact time of at least 10 seconds in addition to the contact time in said first mixing chamber;
collecting said mixture, then
centrifuging said collected mixture so as to recover the clarified supernatant, comprising said peptide of interest.

### BRIEF DESCRIPTION OF THE DRAWINGS

FIG. 1 shows the simplest version of the automated extraction method.

FIG. 2 shows the automated extraction method including an acidification step via a T-connection.

FIG. 3 shows the automated extraction method including an acidification step via a second mixing chamber.

FIG. 4 shows two examples of purification of two different proteins expressed in the periplasmic space, each having a size of approximately 15 kDa.

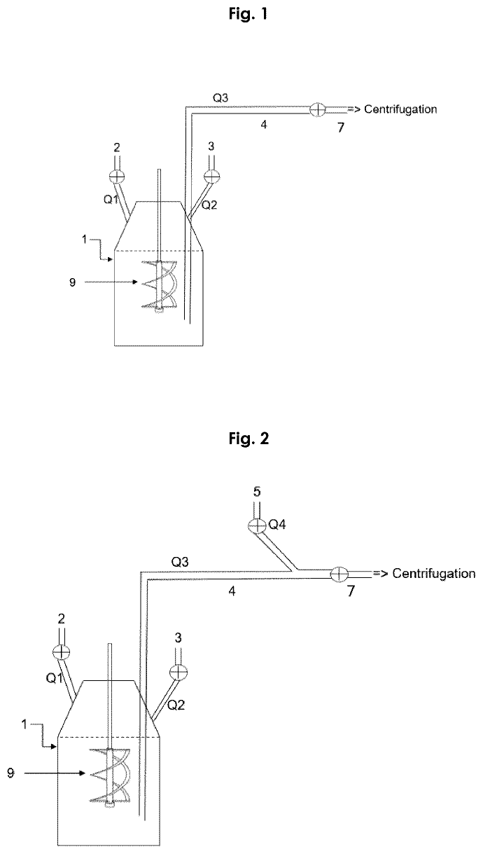

### DETAILED DESCRIPTION OF AN EMBODIMENT OF THE INVENTION

A first aspect of this invention relates to an automated method for extracting a peptide of interest artificially secreted into the periplasmic space of a Gram-negative bacterium comprising the successive steps of:
suspending the bacteria in a hypertonic solution,
pumping 2 the suspension in a hypertonic medium into a first mixing chamber 1, at a constant and calibrated flow rate Q1;
simultaneously pumping at a constant and calibrated flow rate Q2 a hypotonic solution 3 into the mixing chamber 1, so that the mixture containing the cells, the hypertonic solution and the hypotonic solution remains for at least 10 seconds in the mixing chamber 1, so as to ensure breakage of the external membrane of the bacteria by osmotic shock while preserving the integrity of the internal membrane of the bacteria;
pumping the mixture to draw it from the mixing chamber 1 towards a pipe 4, at a constant and calibrated flow rate Q3, so that the volume of the mixture in the mixing chamber 1 makes it possible to ensure the desired residence time in the mixing chamber 1, the pipe 4 having a length and a diameter so as to ensure a contact time of at least 10 seconds in addition to the contact time in the first mixing chamber 1;
collecting the mixture 7, then
centrifuging the collected mixture so as to recover the clarified supernatant, comprising the peptide of interest.

This makes it possible to treat large quantities of cells while ensuring the integrity of the internal membrane and ensuring a purity at least equivalent to that obtained by a batch method, even if practised on a much more modest scale.

The residence time in the mixing chamber 1 is preferably between 10 seconds and 10 minutes, advantageously between 30 seconds and 5 minutes, for example between 1 and 3 minutes, such as approximately 2 minutes.

Similarly, the residence time in the pipe 4 is preferably between 10 seconds and 10 minutes, advantageously between 30 seconds and 5 minutes, for example between 1 and 3 minutes, such as approximately 2 minutes.

Preferably, this method further comprises the step of adding an acidic solution 5 to the solution comprising the cell suspension, the hypertonic solution and the hypotonic solution (before the centrifugation step), while (substantially) preserving the solubility of the peptide of interest.

Indeed, although the usual methods are commonly carried out at neutral or alkaline pHs (in any case rarely at pH<6.5, or even<6), which are compatible with those of the downstream chromatographic steps, which is considered advantageous, the inventors noted that acidification makes it possible to easily increase the level of purity of the peptide to be isolated, which is very advantageous.

This step is advantageous only for peptides that remain (substantially) soluble in an acidic medium. Thus, the method preferably comprises a preliminary step of determining the solubility of the peptide in an acidic medium. This allows the peptide of interest to be (substantially) preserved in a solution while applying the most pronounced acidification possible, which allows the precipitation of as many contaminants as possible. In the context of this invention, it is preferably understood by “to (substantially) preserve the peptide of interest in a solution” that at least 50% (by weight), at least 60% by weight, at least 65%, at least 70, 75, 80, 85, 90, or even 95, 96, 97, 98, 99 or substantially all of the peptide of interest is preserved (maintained) in a solution. An advantageous manner of carrying out the measurement is via acrylamide gel electrophoresis followed by specific staining compatible with quantification. Thus, preferably, the acidic solution is added so as to obtain a final pH of between 3.0 and 6.0, preferably between 4.0 and 5.0, even more preferably between 4.2 and 4.8 (depending on the pH which is compatible with the solubility of the peptide of interest to be purified, with a pH as acidic as possible being preferred). The pH, preferably, is continuously monitored by a probe, and is adjusted to the desired value by modulating the flow rate Q4.

According to an alternative (FIG. 2), the step of acidifying the solution is carried out directly at the level of the pipe 4 receiving the flow Q3 (or a periplasmic extract obtained differently, or even a cell lysate, see below) via the continuous addition to said flow Q3 of a flow Q4 of an acid solution 5, preferably so as to obtain a final pH of between 3.0 and 6.0, preferably between 4.0 and 5.0, even more preferably between 4.2 and 4.8. Thus the flow Q4 is not necessarily constant, but can vary (slightly);

in other words, precise control of the pH is, according to the invention, more important than a slight variation in the final volume 7.

According to the other alternative (FIG. 3), the step of adding the acid solution 5 is carried out in a second mixing chamber 6, the second mixing chamber 6 receiving the flow rate Q3 and a flow rate Q4 of the acid solution 5, so as to produce a homogeneous mixture, this mixture being drawn off from the second mixing chamber 6 at a constant flow rate, so as to ensure a volume allowing the desired residence time in the second mixing chamber 6.

As in the first alternative, preferably, the acid solution has a pH of between 3.0 and 6.0, such as between 4.0 and 5.0, even more preferably between 4.2 and 4.8.

Preferably, the acidifying solution comprises (consists essentially of) an acid, preferably acetic acid, preferably at a concentration of between 0.2 M and 3.0 M, such as about 0.5 M. A concentrated solution is advantageous because it avoids increasing volumes too much. However, the pH is more difficult to adjust in this case and the viscosity of the solution increases, which makes mixing difficult. Other acids may be advantageously used, for example hydrochloric acid, citric acid or phosphoric acid.

Preferably, the solutions are added and drawn off via the bottom of the first and/or second mixing chamber.

Advantageously, the flows Q1 and Q2 are mixed via the turbulence caused by the arrival of the flow Q1 and/or Q2 in the mixing chamber 1.

Preferably, the flows Q1 and Q2 are mixed via mechanical stirring 9 in the first mixing chamber 1, this mechanical stirring being calibrated so as not to cause the breakage of the internal membranes of the bacteria.

Alternatively, or in addition, the flows Q3 and Q4 are mixed via mechanical stirring 8 in the second mixing chamber 6, and this mechanical stirring 8 is calibrated.

When the second mixing chamber is not used, the flows Q3 (or a periplasmic extract obtained differently) and Q4 are mixed by the turbulence caused at the connection. This advantageously allows rapid acidification.

Preferably, and in particular in connection with the addition of an acidification solution, the hypertonic solution and/or the hypotonic solution of the method are at a pH between 3.0 and 5.0, preferably between 3.5 and 4.0.

If the peptide to be purified is compatible with such acidification, the use of solutions causing acid osmotic shock will simplify the acidification effort downstream.

Furthermore, surprisingly, the inventors noted that the internal membranes of the bacteria were better preserved in acidic, or even strongly acidic, conditions, while the external membranes remained just as sensitive to osmotic shock, regardless of the pHs tested.

Advantageously, the pH of the hypertonic solution and/or the pH of the hypotonic solution is set by a buffer with a concentration of between 10 and 100 mM, preferably between 15 and 50 mM, advantageously around 20 mM, for example a 20 mM acetic acid (acetate) buffer.

Thus, this buffer is relatively weak and the pH of the medium, after breakage of the external membrane of the bacteria, may be higher, but this may be corrected and the desired pH may advantageously be precisely set during subsequent acidification, if it takes place.

A related aspect of this invention is a method for purifying peptides artificially secreted into the periplasmic space of Gram-negative bacteria comprising the successive steps of:
applying an osmotic shock to said bacteria, so as to specifically release the periplasmic space,
applying an acidification to said released periplasmic space, so as to specifically precipitate the bacterial contaminants, but not the peptide of interest and
obtaining the purified peptide by sedimentation and/or by centrifugation.

As mentioned above, this method advantageously comprises the preliminary step of determining the solubility of the peptide of interest in different acidic solutions and of using a solution sufficiently acidic to precipitate the bacterial contaminants while maintaining the peptide of interest in a solution. In practice, this method is also advantageous even if a minor proportion of the peptide of interest is no longer in a solution due to the acidification, provided that a larger proportion of contaminating proteins is removed by this acidification. Thus, in the context of this invention, “keeping the peptide of interest in a solution” preferably means that at least 50% (by weight), at least 60% by weight, at least 65%, at least 70, 75, 80, 85, 90, or even 95, 96, 97, 98, 99 or substantially all of the peptide of interest is kept in a solution. An advantageous way of carrying out the measurement is via acrylamide gel electrophoresis followed by specific staining compatible with quantification.

Preferably, the pH of the acidification solution (or of the solution after addition of the acidification solution) in this method is between 3.0 and 6.0, preferably between 4.0 and 5.0 (the pH is as acidic as possible while ensuring the solubility of the peptide of interest).

Preferably, the acid solution added in this method comprises an acid, preferably selected from acetic acid, citric acid, hydrochloric acid and phosphoric acid at a concentration preferably between 0.2 M and 3 M, preferably between 0.3 M and 1.5 M, even more preferably between 0.4 M and 1.0 M.

Preferably, in this method, the acid solution is added continuously and/or the homogenisation of the solution comprising the periplasmic extract with the acid solution is rapid. For example, the mixing of the two solutions is done continuously via “T” or “Y” connections, which makes it possible to cause local turbulence, conducive to rapid homogenisation.

Alternatively, the acid solution 5 can advantageously be added via a (second) mixing chamber 6. This mixing chamber 6 receiving the flow rate Q3 (which is, in this alternative, a periplasmic extract not necessarily obtained from the first mixing chamber 1, such as a periplasmic extract obtained by a batch method or via a continuous method) and a flow rate Q4 of the acid solution 5, so as to produce a homogeneous mixture, this mixture being drawn off from the (second) mixing chamber 6 at a constant flow rate, so as to ensure a volume allowing the desired residence time in the (second) mixing chamber 6. Calibrated mechanical stirring can advantageously be applied so as to accelerate homogenisation.

The inventors noted that the use of an acid, rather than a buffer mixture (the acid and a base, so as to better set a precise pH), was advantageous because it limited the variations in the conductivity of the composition after mixing.

In line with the above, preferably, the hypertonic and/or hypotonic solutions used for the osmotic shock of this method are acidic, preferably at a pH between 3.0 and 5.0, more preferably between 3.5 and 4.0.

Advantageously, the pH of the hypertonic solution and/or the pH of the hypotonic solution is set by a buffer with a concentration of between 10 and 100 mM, preferably between 15 and 50 mM, advantageously around 20 mM, for example a 20 mM acetic acid (acetate) buffer.

According to one variant, this acidification method is carried out directly on a lysate of Gram-negative bacteria expressing a recombinant peptide (of interest), rather than on the periplasmic extract.

The two methods above, and the variant of the second method, have been successfully applied to CRM197, as well as to other peptides remaining soluble under acidic conditions.

For example, the peptide of interest (or recombinant, or expressed in the periplasmic space) is advantageously chosen from an enzyme, an antibody fragment (preferably a “single domain antibody” and/or “immunoglobulin single variable domain”), a coagulation factor and an epitope and/or, advantageously, this peptide has a molecular weight of between 5 and 200 kDa, preferably between 10 and 50 kDa, preferably between 12 and 20 kDa.

Other characteristics and advantages of this invention will be drawn from the non-limiting description below, and with reference to the drawings and the examples.

### EXAMPLES

It is understood that this invention is in no way limited to the embodiments described above and that many modifications can be made without departing from the scope of the appended claims.

### Comparative Example

The inventors used 2×30 g of frozen Escherichia coli cell paste expressing a protein of interest (CRM197; SEQ ID NO:1) in the periplasmic space. The paste was thawed at room temperature and then re-suspended in two flasks each time with 60 ml of a hypertonic buffer (20 mM sodium acetate, 5 mM EDTA; 20% (w:w) sucrose; pH 3.6) and gently mixed (manually) for about 10 minutes, until the aggregates disappeared.

In each flask containing the suspension, this was then diluted with 210 ml of a cold hypotonic buffer (5 mM MgCL₂, 20 mM sodium acetate, pH 3.6) and gently mixed (manually) for 30 seconds. Then, in one of the conditions, the pH of the suspension was adjusted by adding an acetic acid solution, so as to obtain a final pH of 4.5. Both conditions were then centrifuged for 20 minutes at 8000 rpm at 4° C. in an Avanti centrifuge.

The supernatant, which contains the periplasmic extract, is recovered and filtered at 0.2 μm.

The inventors observed to their surprise that this brief osmotic shock time was sufficient and that acidification increased the purity of the protein of interest (from 50% to 65%) by causing the precipitation of E. coli contaminating proteins. The protein of interest did not precipitate at this pH.

### Example 1

Periplasmic extraction in automated mode. Another batch of Escherichia coli cellular paste expressing a protein of interest (CRM197; SEQ ID NO:1) in the periplasmic space was used, this time for automated periplasmic extraction.

The thawed paste (450 g) at room temperature is incubated for 15 minutes in 900 ml of a hypertonic solution (20 mM sodium acetate, 5 mM EDTA; 20% (w:w) sucrose; pH 3.6) with gentle mechanical stirring.

The sample is then pumped into a mixing chamber at a fixed and constant flow rate Q1 and, at the same time, the cold hypotonic solution is added at another fixed and constant flow rate Q2, so that the entire hypotonic solution (3.1 l) is pumped at the same time as the hypertonic suspension comprising the cells. The hypotonic solution is 5 mM MgCL₂, 20 mM sodium acetate, pH 3.6. Then, when a volume allowing a desired contact time is reached in the mixing chamber, the mixture is pumped at a constant flow Q3 which is the sum of flows Q1 and Q2 (the volume in the mixing chamber therefore remains substantially constant, until the mixing chamber is emptied) and this mixture passes into a pipe of determined diameter and length, so as to ensure a residence time of 30 seconds. The pipe leads to a second mixing chamber where an acetic acid solution (HAc 0.5M; pH=4.5) is pumped in at a suitable flow rate, so as to ensure a pH of 4.5. The pH is monitored continuously. The residence time of the mixture in the acidification chamber is 30 seconds and, once the volume allowing this residence time is reached, a pump extracts the mixture containing the acidified sample at a flow rate Q5 (Q5=Q3+Q4). Then, the mixture is centrifuged and the supernatant, which contains the clarified periplasmic extract, is retained. A purity as good as in the manual method with acidification, 65%, is obtained. However, this method allows significantly larger quantities to be treated, 15 times more in this example, while ensuring a controlled process, which guarantees its reproducibility, even when very large quantities are treated.

The inventors also tested the same periplasmic extraction system, but without the addition of acetic acid after the osmotic shock. The purity was significantly lower, less than 50%. However, again, this automated method allows large quantities to be treated. The protein of interest can, if necessary, be purified via one or more chromatographies.

### Example 2

According to one variant, the second mixing chamber is omitted, and the acidification solution (0.5 M HAc) is added directly to the end of the piping containing the mixture of hypertonic and hypotonic solutions through which the Q3 flow passes, so as to obtain a final pH of 4.5; this is done via a “T” connector and the homogeneity of the mixture is ensured by adapting the diameter of the ducts, the contact time being ensured by further adapting the length of the pipes.

### Example 3—Automation and Generalisation

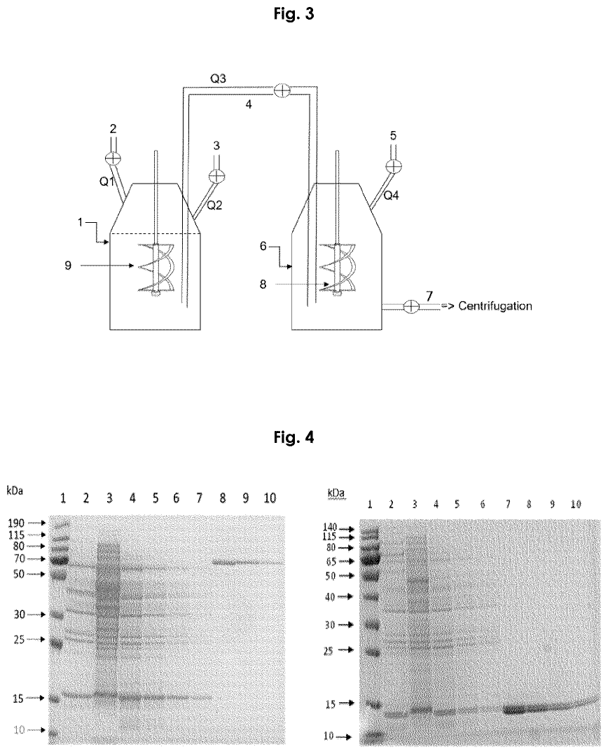

The inventors then applied the technology for the purification of two peptides fermented and then exported into the periplasmic space of E. coli. These two peptides are quite similar in size (15.5 and 12.6 kDa, FIGS. 4A and 4B), but were less well produced and/or exported into the periplasmic space than the protein in the examples above, which has a direct effect on purity.

FIG. 4(A): column 1, molecular weight markers; column 2: old batch (control); column 3: periplasmic extract according to the invention; Columns 4-7, periplasmic extract after acidification (successive dilutions 1×, 2×, 4× and 8×). Column 8-10: bovine serum albumin (BSA) reference; 1 μg; 0.5 μg and 0.25 μg.

FIG. 4(B): Column 1, molecular weight markers; Column 2: old batch (control); Column 3: periplasmic extract according to the invention; Columns 4-6, periplasmic extract after acidification (successive dilutions 1×, 2× and 4×). Column 7-10: previously purified reference protein (4 μg; 2 μg; 1 μg and 0.5 μg).

The first peptide retains almost total solubility under acidic conditions.

This peptide represents 6.9% of the periplasmic space (column 3). When acid precipitation was performed (column 4), this peptide now represents 18.4%.

The second peptide has reduced solubility under acidic conditions.

This peptide represents 15% of the periplasmic space. When acid precipitation has been performed, this peptide now represents 28% of the total, although approximately 35% of the peptide has precipitated and is therefore lost. However, since the method is simple to carry out, and does not involve expensive components, the inventors consider that the acid precipitation step remains advantageous, even for this peptide.

1. An automated method for extracting a peptide of interest artificially secreted into the periplasmic space of a Gram-negative bacterium comprising the successive steps of:
suspending said bacteria in a hypertonic solution,
pumping said suspension in a hypertonic medium into a first mixing chamber, at a constant and calibrated flow rate Q1;
simultaneously pumping at a constant and calibrated flow rate Q2 a hypotonic solution into said mixing chamber, so that the mixture containing said cells, the hypertonic solution and the hypotonic solution remains for at least 10 seconds in said mixing chamber, so as to ensure breakage of the external membrane of said bacteria by osmotic shock while preserving the integrity of the internal membrane of said bacteria;
pumping said mixture to draw it from said mixing chamber to a pipe, at a constant and calibrated flow rate Q3, such that the volume in said first mixing chamber makes it possible to ensure the desired residence time in said first mixing chamber, said pipe having a length and a diameter so as to ensure a contact time of at least 10 seconds in addition to the contact time in said first mixing chamber;
collecting said mixture, then
centrifuging said collected mixture so as to recover the clarified supernatant, comprising said peptide of interest.

2. The method according to claim 1, further comprising the step of adding an acidic solution to the solution comprising the cell suspension, the hypertonic solution and the hypotonic solution while maintaining the solubility of the peptide of interest, preferably wherein the solubility of the peptide in an acidic medium has been predetermined.

3. The method according to claim 2, wherein the step of acidifying the solution is carried out directly at the level of the pipe receiving the flow Q3 via the continuous addition to said flow Q3 of a flow Q4 of an acidic solution, preferably so as to obtain a final pH of between 3.0 and 6.0, preferably between 4.0 and 5.0, even more preferably between 4.2 and 4.8.

4. The method according to claim 2, wherein the step of adding an acid solution is carried out in a second mixing chamber, said second mixing chamber (6) receiving the flow rate Q3 and a flow rate Q4 of said acid solution, so as to produce a homogeneous mixture, said mixture being drawn off from said second mixing chamber (6) at a constant flow rate, so as to ensure a volume allowing the desired residence time in said second mixing chamber (6), said acid solution preferably having a pH between 3.0 and 6.0, such as between 4.0 and 5.0, even more preferably between 4.2 and 4.8.

5. The method according to claim 2, wherein the acid solution is acetic acid, preferably at a concentration between 0.2 M and 3.0 M, such as approximately 0.5 M.

6. The method according to claim 1, wherein the solutions are added and drawn off via the bottom of the first (1) and/or the second (6) mixing chamber.

7. The method according to claim 1, wherein the flows Q1 and Q2 are mixed via the turbulence caused by the arrival of the flow Q1 and/or Q2 in the mixing chamber.

8. The method according to claim 1, wherein the first (1) and/or the second (2) mixing chamber comprises mechanical stirring (8, 9), said mechanical stirring (8, 9) being calibrated so as not to cause the breakage of the internal membranes of the bacteria.

9. The method according to claim 1, wherein the hypertonic solution and/or the hypotonic solution are at a pH between 3.0 and 5.0, preferably between 3.5 and 4.0.

10. The method according to claim 9, wherein the pH of the hypertonic solution and/or the pH of the hypotonic solution is set by a concentration buffer between 10 and 100 mM, preferably between 15 and 50 mM, advantageously around 20 mM.

11. The method according to claim 9, wherein the hypertonic and/or hypotonic solution comprises a 20 mM acetic acid (acetate) buffer.

12. A method for purifying peptides artificially secreted into the periplasmic space of Gram-negative bacteria, comprising the successive steps of:
applying an osmotic shock to said bacteria, so as to specifically release the periplasmic space,
applying an acidification of said released periplasmic space, so as to specifically precipitate the bacterial contaminants, but not the peptide of interest and
obtaining the purified peptide by sedimentation and/or by centrifugation.

13. A method for purifying recombinant peptides expressed in Gram-negative bacteria, comprising the successive steps of:
lysing said bacteria,
applying an acidification of said bacteria lysate comprising said recombinant peptide, so as to specifically precipitate the bacterial contaminants, but not said recombinant peptide, and
obtaining the purified peptide by sedimentation and/or by centrifugation.

14. The method according to claim 12, comprising the preliminary step of determining the solubility of the peptide of interest in different acidic solutions and using a sufficiently acidic solution to precipitate the bacterial contaminants while maintaining the peptide of interest in a solution.

15. The method according to claim 12, wherein the pH of the acidification solution is between 3.0 and 6.0, preferably between 4.0 and 5.0.

16. The method according to claim 12, wherein the acidic solution comprises an acid, preferably selected from acetic acid, citric acid, hydrochloric acid and phosphoric acid, at a concentration preferably between 0.2 M and 3 M, preferably between 0.3 M and 1.5 M, even more preferably between 0.4 M and 1.0 M.

17. The method according to claim 12, wherein the acidic solution is added continuously to the periplasmic extract, wherein a flow comprising the periplasmic extract is continuously mixed with a flow comprising the acidic solution.

18. The method according to claim 12, wherein the acidic solution (5) is added via a mixing chamber (6), preferably with calibrated mechanical stirring (8).

19. The method according to claim 12, wherein the hypertonic and/or hypotonic solutions used for the osmotic shock are acidic, preferably at a pH between 3.0 and 5.0, more preferably between 3.5 and 4.0 and/or preferably via an acidic buffer of concentration between 10 and 100 mM, preferably between 15 and 50 mM.

20. The method according to claim 1, wherein the peptide is selected from an enzyme, an antibody fragment, a coagulation factor, an epitope and CRM197 and/or wherein said peptide has a molecular weight of between 5 and 200 kDa, preferably between 10 and 50 kDa, preferably between 12 and 20 kDa.
